(19) United States
(12) Patent Application Publication
(10) Pub. No.: US 2026/0316347 A1
(43) Pub. Date: Oct. 15, 2026

(54) A MOULD SYSTEM FOR MANUFACTURING DISSOLVABLE MICRONEEDLE PATCHES

(71) Applicant: Chee Yen LIM, Singapore (SG)

(72) Inventor: Chee Yen LIM, Singapore (SG)

(21) Appl. No.: 19/483,244

(22) PCT Filed: Jul. 19, 2023
(86) PCT No.: PCT/SG2023/050507

§ 371 Date: Nov. 11, 2025

(51) Int. Cl. B29C 39/26; A61M 37/00; B29C 33/00; B29C 39/42; B29L 31/00

(52) U.S. Cl. CPC B29C 39/26; A61M 37/0015; B29C 39/42; A61M 2037/0053; B29C 2033/0094; B29K 2715/006; B29K 2883/00; B29K 2995/0093; B29L 2031/7544

(57) ABSTRACT

The present invention involves a mould system for manufacturing dissolvable microneedle patches, comprising (1) a flat silicone rubber mould 100 which comprises a plurality of microneedle cavities 110 on one of its surfaces, and (2) a mask 420, which comprises a mask surface 430 and at least a through hole 440 having the shape of the dissolvable microneedle patch, as shown in FIG. 4.

### FIELD OF THE INVENTION

The present invention generally relates to a mould system for manufacturing dissolvable microneedle patches. More particularly, the present invention pertains to a mould system for manufacturing microneedle patches, comprising (1) a flat silicone rubber mould comprising a plurality of microneedle cavities on one of its surfaces and (2) a mask that is placed on the silicone rubber mould to define the shape of the dissolvable microneedle patches.

### BACKGROUND OF THE INVENTION

Over the past decade, microneedle technology has been increasingly applied in the treatment of skin conditions. The decreased likelihood of causing pain, infection, and injury makes microneedles a great platform for self-administration of drugs. In recent years, drug-loaded dissolving microneedles have received extensive attention in transdermal drug delivery. In contrast to drug-coated microneedles, in which drug is coated on the surface of solid, non-dissolvable microneedles, these dissolvable microneedles do not impose the risk of having foreign material residing in the skin if broken. They can also encapsulate sensitive biomolecules within the needle tips and shafts so that a specific dosage of drugs can be delivered to the skin. They certainly possess a higher drug-loading capacity compared to the coated solid microneedles.

Dissolvable microneedle patches are desired to be flexible and large in patch size to treat skin conditions, such as wounds, eczema, depigmentation, etc. which normally involves a large area of the skin. The shape of the dissolvable microneedle patches is also variable, they can be rectangular, squarish, round, crescent or ellipse shape for various parts of the body. Hence, to make these microneedle moulds in various shapes and sizes become challenging and expensive.

Most dissolvable microneedles involve using hydrogels as the raw material; one example is hyaluronic acid, which is quite viscous when its concentration and/or molecular weight is high. Therefore, normally these materials are mixed at low concentration for casting over the silicone rubber moulds. For a sizeable patch, the volume of the material solution may be 0.05 mL to 5 mL. As shown in FIG. 1 (prior art), a conventional way for containing the material solution is to have a liquid catchment 105 on the silicone rubber mould 100. This liquid catchment 105 is formed by raising the surrounding non-moulding area 115, which has a boundary that defines the shape of the dissolvable microneedle patch. A liquid catchment of several millimeters deep is normally required to contain the material solution within the mould until it dries up and turns into a dissolvable microneedle patch. FIG. 2 (prior art) shows the drying state of the material solution on the silicone rubber mould 100. As the material solution 210 diminishes, some liquid will reside on the wall of the liquid catchment 220 and form a flashing or burr 230 around the perimeter of the silicone rubber mould. An extra cutting step is required to trim the formed dissolvable microneedle patches into shape. This requirement poses another challenge to the manufacturing because the cutting may damage the delicate microneedles.

Thirdly, the material solution in the liquid catchment may recede or detach from the wall of the liquid catchment. Let us revisit some surface wetting theory.

The wetting of a solid surface by a liquid is an outcome of the competition between the surface energy of a solid wall and the surface tension of a liquid. When a solid surface has high surface energy and a liquid has low surface tension, the liquid will wet or adhere to the solid surface. Alternatively, when a solid surface has low surface energy and a liquid has high surface tension, the liquid will not wet the solid surface but tries to minimize its surface area by forming a meniscus or sphere.

As shown in FIG. 3(a), at the beginning of the drying stage, the water level of the material solution 210 is high, the surface wetting force 320 of the liquid is larger or equal to the surface tension force 340 of the liquid causing the liquid to wet the wall. After some time, as shown in FIG. 3(b), as the material solution 210 dries up, its volumes reduces and its concentration goes up, the higher concentration increases the surface tension force 340. The concentration will continue to rise during the drying state, which makes the surface tension force 340 to rise continuously until a certain stage where it exceeds the surface wetting force 320, causing the liquid to recede or detach from the wall of the liquid catchment 220, as shown in FIG. 3(c). This phenomenon happens because the liquid's surface tension force prevails and tries to minimize its surface area by forming a meniscus or droplet. The surface wetting force between the liquid and a solid wall is determined by the surface energy of the solid wall, which is constant throughout the phenomenon. Surface wetting will occur when the solid surface has high enough surface energy to compete with the liquid's surface tension. The solid wall of liquid catchment 220 is normally part of the silicone rubber mould, so the wall is slightly hydrophobic or has low surface energy, which contributes to the recession phenomenon.

Here we identify three challenges the prior art imposes to the manufacturing of dissolvable microneedle patches, namely (1) for skin condition treatments, various shapes and sizes of dissolvable microneedle patches are required, these shapes and sizes require too many moulds, which are too expensive and tedious to make; (2) the current liquid catchment is deep and it causes liquid residue on the wall that turns into flashing or burr on the edges of the formed dissolvable microneedle patches; and (3) the material solution may recede or detach from the boundary of the liquid catchment leading to incomplete formation of dissolvable microneedle patches. The present invention seeks to provide technical solutions to overcome these three drawbacks.

### The Technical Solutions of the Invention

The first technical solution, which can be shown in FIG. 4, is to prepare (1) a flat silicone rubber mould 100 having a plurality of microneedle cavities 110 on one of its surfaces and (2) a mask 420 having (a) a mask surface 430 and (b) at least one through hole 440 that has the shape of the dissolvable microneedle patch. The through hole 440 can have any shape such as but not limited to round, square, rectangular, oval, crescent, etc. This mask 420 is placed on the flat silicone rubber mould 100 such that the through hole 440 exposes the microneedle cavities 110 on the flat silicone rubber mould 100. A material solution is dispensed on these through holes 440 so that it covers the through holes 440 and later forms into a dissolvable microneedle patch when dried. Since the flat silicone rubber mould 100 can be used with any new mask every time a new shape or new size is required, the mould cost is drastically reduced.

Now, the second technical solution against the flashing or burr 230 on the wall of the liquid catchment 220 will be explained. As shown in FIG. 2 (prior art) previously, the conventional liquid catchment 220 will cause the material solution to reside and dry, forming the flashing or burr 230 at the edges of the dissolvable microneedle patches. Our solution, which is shown in FIG. 5, involves making mask 420 to be as thin as the dissolvable microneedle patch to eliminate the flashing or burr 230 in FIG. 2. A typical dissolvable microneedle patch may have a thickness ranging from 0.1 mm to 0.5 mm. In addition to the mask's thickness, the mask surface 430 should be hydrophobic so that the smeared liquid will be pushed back to the through hole 440. This is a critical characteristic because as the thickness of the mask is drastically reduced, the mask 420 will not be able to contain the volume of the material solution, and the material solution will overflow to the mask surface 430. Making the mask surface 430 hydrophobic prohibits such overflow thus can effectively contain the material solution within the through hole 440.

Finally, the material solution may recede and detach from the boundary of the through hole or liquid catchment during the drying stage, forming incomplete dissolvable microneedle patches. This liquid recession problem is common in the conventional “liquid catchment” approach as well as the “thin mask” approach in the present invention. An effective solution, which is the third technical solution, to this problem of “liquid recession from the boundary of the through hole” is to maintain a high surface energy on the solid wall by having a non-hydrophobic boundary so that the liquid will clinch on to the boundary during the drying stage and form perfect dissolvable microneedle patches.

Hence, combining all three technical solutions, we will have a mask that (1) can be prepared inexpensively and applied readily to the flat silicone rubber mould, that (2) is as thin as the dissolvable microneedle patch, that (3) has a hydrophobic surface and that (4) has a non-hydrophobic boundary of the through holes on the mask.

### The Closest Prior Arts

A thorough technological review has been conducted for the present invention and we have found no prior art which discloses manufacturing of dissolvable microneedle patches using a mask to determine the shape of the dissolvable microneedle patches. Most prior arts used masks to shape or make the microneedles themselves by exposing or covering the unwanted portion. For example, prior art U.S. Pat. No. 10,899,606B2 disclosed using a mask to cover the microneedles and expose the unwanted material to etching; prior art WO20211066755A2 used a mask and a lens to expose UV ray onto a UV-cured material for forming microneedles; and there are many more prior arts with similar intentions.

### SUMMARY OF THE INVENTION

The present invention involves a mould system for manufacturing dissolvable microneedle patches, comprising (1) a flat silicone rubber mould 100 which comprises a plurality of microneedle cavities 110 on one of its surfaces, and (2) a mask 420, which comprises a mask surface 430 and at least a through hole 440 having the shape of the dissolvable microneedle patch, as shown in FIG. 4.

In the second preferred embodiment, as shown in FIG. 6, the mould system comprises (1) a flat silicone rubber mould 400 which comprises a plurality of microneedle cavities on one of its surfaces, and (2) a mask 420 which is constituted by a top hydrophobic layer 610 and a bottom non-hydrophobic layer 620. The mask 420 is attached to the flat silicone rubber mould 100 through adhering the bottom non-hydrophobic layer 620 the surface comprising microneedle cavities 110. In practice, the top hydrophobic layer 610 is made of plastic film between 0.001 mm to 0.3 mm, which may be further coated with silicone to enhance its hydrophobicity. The material can be PE, PET, APET or other polymers that have the same characteristics. Similarly, the top and bottom non-hydrophobic layer 620 can be a coating of acrylic adhesive between 0.001 mm to 0.3 mm, and the carrier 630 may be any plastic film with thickness 0.001 mm to 0.3 mm. This preferred embodiment may be a conventional double-sided adhesive tape.

In the third preferred embodiment of the present invention, as shown in FIG. 7, the mould system comprises (1) a flat silicone rubber mould 400 which comprises a plurality of microneedle cavities on one of its surfaces, and (2) a mask 420 which is constituted by a top hydrophobic layer 610, a top 615 and bottom 620 non-hydrophobic layers, a central carrier 630, which may be hydrophobic, neutral or hydrophilic. The mask 420 is attached to the flat silicone rubber mould 100 through adhering the bottom non-hydrophobic layer 625 to the surface comprising microneedle cavities 110. In practice, the hydrophobic layer 610 can be any plastic film with thickness 0.01 mm to 0.3 mm, which may be further coated with silicone to enhance its hydrophobicity. The material can be PE, PET, APET or other polymers that have the same characteristics. The non-hydrophobic layer 620 can be a coating of acrylic adhesive between 0.001 mm to 0.3 mm. The second embodiment may be a conventional single adhesive without carrier.

### BRIEF DESCRIPTION OF THE FIGURES

FIG. 1 (prior art) shows a cross section of a prior art's silicone rubber mould 100, which comprises a liquid catchment 105 by raising the surrounding non-moulding area 115.

FIG. 2 (prior art) shows the drying state of the material solution 210 on the silicone rubber mould 100. As the liquid level goes down, some liquid resides on the wall of liquid catchment 220 which turns into flashing or burr 230 when dried.

FIG. 3 shows the surface wetting force 320 and surface tension force 340 of the material solution 210 during the drying stage. (a) when the water volume is high, the surface tension force 340 is smaller or equal to the surface wetting force 320 (wetting prevails); (b) when the water volume reduces due to drying, the surface tension becomes larger than the surface wetting force 320, causing the liquid to recede or detach from the wall of the liquid catchment 220 (surface tension prevails); (c) after recession, the material solution 210 detach form the wall of the liquid catchment 220 and forms incomplete dissolvable microneedle patch.

FIG. 4 shows the first technical solution and the first preferred embodiment of the present invention, comprising (1) a flat silicone rubber mould 100 having a plurality of microneedle cavities 110 on one of its surfaces, and (2) a mask 420 comprising a mask surface 430 and at least one through hole 440 having the shape of dissolvable microneedle patch.

FIG. 5 shows the second technical solution of the present invention, comprising a thin mask 420, which comprises a hydrophobic mask surface 430 and a through hole 440 having the shape of the dissolvable microneedle patch.

FIG. 6 shows the second preferred embodiment of the present invention, comprising (1) a flat silicone rubber mould 100 having a plurality of microneedle cavities 110 on one of its surfaces; and (2) a multi-layered mask 420 having mask surface 430 and at least a through hole 440, the mask 420 is constituted by a top hydrophobic layer 610, and a bottom non-hydrophobic layer 620.

FIG. 7 shows the third preferred embodiment of the present invention, comprising (1) a flat silicone rubber mould 100 having a plurality of microneedle cavities 110 on one of its surfaces; and (2) a multi-layered mask 420 having mask surface 430 and at least a through hole 440, the mask 420 is constituted by a top hydrophobic layer 610, a top 615 and a bottom 620 non-hydrophobic layers, and a central carrier 630.

FIG. 8 shows the meniscus 800 of the material solution 210 that is formed by using a thin mask with hydrophobic mask surface 430.

FIG. 9 shows the manufacturing steps of manufacturing dissolvable microneedle patches using the first preferred embodiment of the present invention.

### DETAILED DESCRIPTION OF THE INVENTION

It is the objective of the present invention to provide a low-cost mould system for the manufacturing of large sized, variable-shaped dissolvable microneedle patches for skin condition treatments. In this section we will explain the spirit of the inventive concept of the present invention through four preferred embodiments of the present invention.

### Example 1

### Elimination of Flashing at the Edges of Dissolvable Microneedle Patches

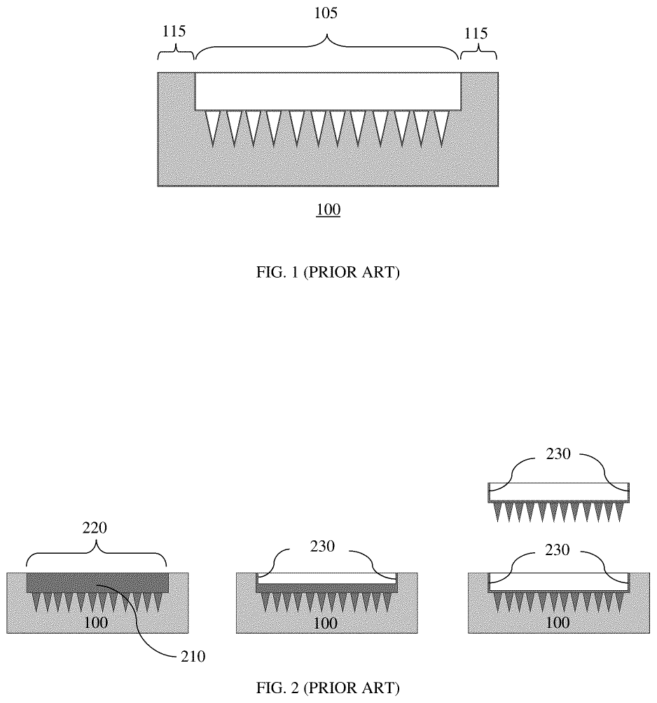

The prior art uses the “liquid catchment” approach in which a wall is formed around the microneedle cavities to contain the material solution as it dries up. As an example, if the liquid catchment area is 10 mm² and the volume of the material solution is 20 μl or 20 mm³, the depth of the liquid catchment required is at least 2 mm. As the material solution fills up the liquid catchment, the solution makes contact with the wall and resides on the wall. The material solution evaporates and eventually dries up. The 2 mm high liquid residue will dry and turn into flashing or burr around the dissolvable microneedle patch, as shown in FIG. 2. As the material solution fills up the liquid catchment, the solution makes contact with the wall and resides on the wall. As explained previously, the present invention replaces the “liquid catchment” approach with a “thin mask” approach in which there is no wall for liquid residue and flashing/burr to form.

The first preferred embodiment of the present invention involves a mould system comprising (1) a flat silicone rubber mould 100, which comprises a plurality of microneedle cavities 110 on one of its surfaces; and (2) a mask 420, which comprises a mask surface 430 and at least a through hole 440 having the shape of the dissolvable microneedle patch. Mask 420 can be several millimeter thick or can be made as thin as possible to eliminate the boundary wall of the liquid catchment thus preventing the formation of flashing or burr 230. The thickness of the mask 420 is in the range of 0.01 mm to 0.3 mm, typically. It is preferrable that the thickness of the Mask 420 is made the same as the thickness of the dissolvable microneedle patches where possible, in this way, there is no wall to accumulate any liquid residue and there will not be any flashing or burr. A typical thickness of the dissolvable microneedle patch is between 0.05 mm to 0.2 mm, beyond which the patch may be too rigid to flex and below which the patch may be too fragile to handle. For this thickness range, the mask 420 can be prepared by die-cutting or punching thin materials. Plastic films or composite plastic films, i.e., multi-layered plastic films make good candidates for the mask. In practice, a double-sided adhesive tape will have the required thickness which is also readily applicable to the flat silicone rubber mould.

### Example 2

### Containment of Material Solution Using Hydrophobic Mask Surface

The required volume of the material solution for making a dissolvable microneedle patch depends on many factors, including the molecular weight of the material, the concentration and the thickness of the patch, among other factors. Through our experience, a common volume to area rate is 1.0 to 10.0 microliter/mm² for certain molecular weight and thickness. As we replace the liquid catchment of the mould with a thin mask, the material solution may overflow out of the boundary of the through holes 440 and flood the mask surface 430. This can be prevented by having a hydrophobic mask surface 430, as explained in the following example shown in FIG. 8.

For example, the mask 420 may be 0.1 mm thick and its through hole area is 10 mm²; the volume of the material solution is 20 μl or 20 mm³. So, after fully covering the through hole's area of 10 mm², the water level is 2 mm high. This 2 mm high water level would require at least a liquid catchment of 2 mm high for containing the liquid within the through hole 440, with a mask's thickness of 0.1 mm, the liquid might overflow out of the through hole 440, as shown in FIG. 8(a).

The wetting of a solid surface by a liquid is an outcome of the competition between the surface energy of a solid wall and the surface tension of a liquid. When a solid surface has high surface energy and a liquid has low surface tension, the liquid will wet or adhere to the solid surface. Alternatively, when a solid surface has low surface energy and a liquid has high surface tension, the liquid will not wet the solid surface but tries to minimize its surface area (normally forming a meniscus or sphere).

We can solve the problem making the mask surface 430 hydrophobic (or lowering its surface energy), thus making the surface tension of the liquid prevails, causing the liquid to minimize its surface area by pulling back the liquid to form a meniscus 800 at the boundary of the through hole 440, as shown in FIG. 8. This meniscus 800 is very stable and will remain contained throughout the drying stage.

The second preferred embodiment of the present invention involves a mould system comprising (1) a flat silicone rubber mould 100 having a plurality of microneedle cavities on one of its surfaces, and (2) a mask 420 having a mask surface 430 which is hydrophobic and having at least a through hole 440 having the shape of the dissolvable microneedle patches. Mask 420 is made as thin as possible to eliminate the boundary wall of the liquid catchment thus preventing the formation of flashing or burr 230. The thickness of the mask 420 is in the range of 0.01 mm to 0.3 mm, typically. It is preferrable that the thickness of the Mask 420 is made to have the same the thickness as of the dissolvable microneedle patches where possible, in this way, there is no wall to accumulate any liquid residue and there will not be any flashing or burr. A typical thickness of the dissolvable microneedle patch is between 0.05 mm to 0.2 mm, beyond which the patch may be too rigid to flex and below which the patch may be too fragile to handle. For this thickness range, the mask 420 can be prepared by die-cutting or punching thin hydrophobic materials. Plastic films or composite plastic films, i.e., multi-layered plastic films are naturally hydrophobic, which make good candidates for the mask. In practice, double-sided or single-sided adhesive tapes have the required thickness which is also readily applicable to the flat silicone rubber mould. Other common ways to make a surface hydrophobic include coating the surface with a layer of hydrophobic material, e.g. silicone, and surface treatment such as plasma treatment to change the surface's property to become hydrophobic.

### Example 3

### Prevention of Liquid Recession From the Boundary of the Through Hole

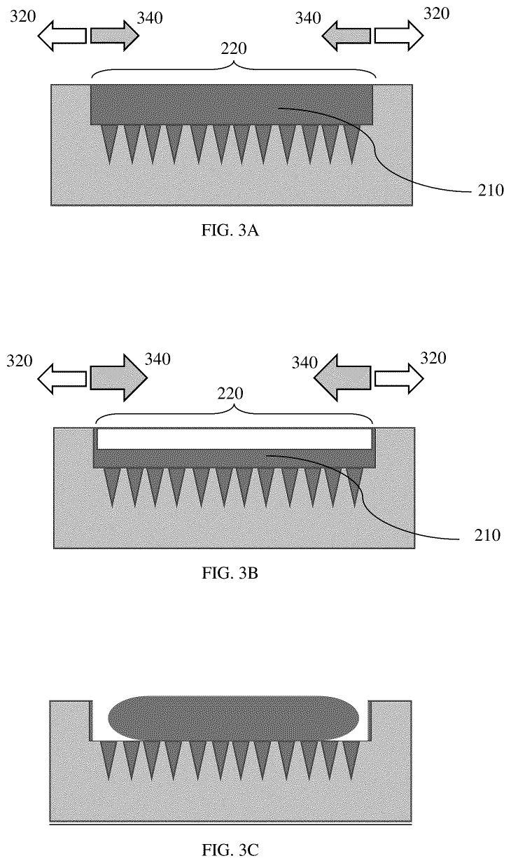

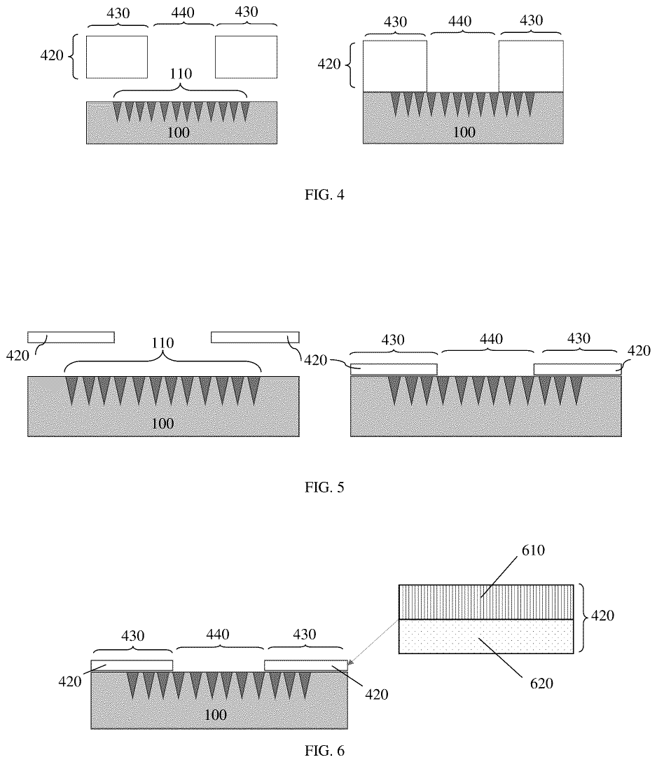

As mentioned earlier, the wetting of a solid surface by a liquid is an outcome of the competition between the surface energy of a solid wall and the surface tension of a liquid. When a solid surface has high surface energy and a liquid has low surface tension, the liquid will wet or adhere to the solid surface. Inversely, when a solid surface has low surface energy and a liquid has high surface tension, the liquid will not wet the solid surface but tries to minimize its surface area by forming a meniscus or sphere. In our case, the liquid catchment on the conventional silicone rubber mould in FIG. 3(a) has low surface energy, so the adhesion of the material solution to the wall of the liquid catchment is not strong and the material solution is loosely adhered to the wall. The material solution will dry up and volume reduction will cause its concentration to rise, thereby increasing its surface tension. As soon as the concentration of the material solution rises to a threshold, causing the surface tension of the material solution to exceed the surface energy of the solid wall, the material solution may recede or detach from the solid wall and form an irregular meniscus, as shown in FIG. 3(c).

To solve this recession problem, we make the solid wall non-hydrophobic or use a material with high surface energy. This is achieved by having a multi-layered mask. The first and the second preferred embodiments will not work because a hydrophobic mask, either by material's natural surface property or by surface treatments, will have the boundary of the through hole also hydrophobic, making the mask prone to liquid recession/detachment problem.

FIG. 6 shows the third preferred embodiment of the present invention, comprising (1) a flat silicone rubber mould 100 having a plurality of microneedle cavities 110 on one of its surfaces; and (2) a multi-layered mask 420 having mask surface 430 and at least a through hole 440, the mask 420 is multi-layered and is constituted by a top hydrophobic layer 610, and a bottom non-hydrophobic layer 620, wherein the bottom non-hydrophobic layer 620 is attached to the flat silicone rubber mould 100 via one of its surfaces that comprises a plurality of microneedle cavities 110.

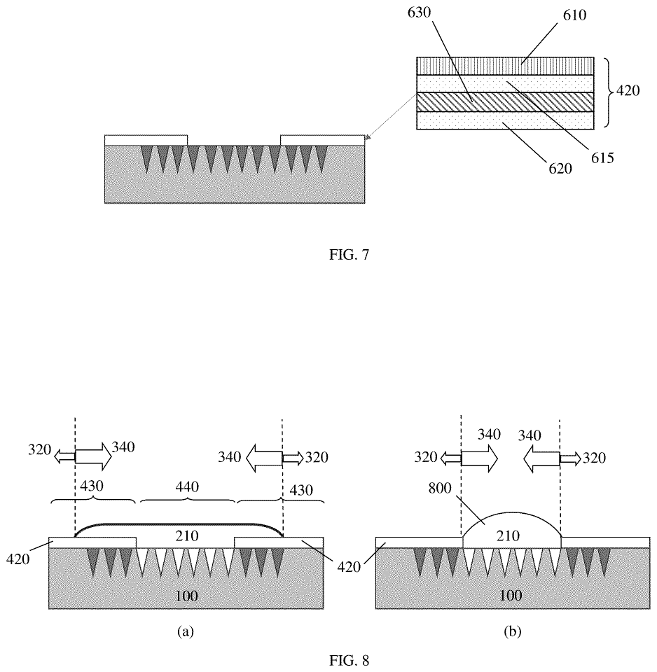

FIG. 7 shows the fourth preferred embodiment of the present invention, comprising (1) a flat silicone rubber mould 100 having a plurality of microneedle cavities 110 on one of its surfaces; and (2) a multi-layered mask 420 having mask surface 430 and at least a through hole 440, the mask 420 is multi-layered and is constituted by a top hydrophobic layer 610, a top 615 and a bottom 620 non-hydrophobic layers, and a central carrier 630 which is sandwiched between the top and bottom non-hydrophobic layers, wherein the bottom non-hydrophobic layer 620 is attached to the flat silicone rubber mould 100 via one of its surfaces that comprises a plurality of microneedle cavities 110.

In the above third and fourth preferred embodiments which comprises a hydrophobic mask surface 430, we can have at least a non-hydrophobic layer below the hydrophobic mask surface 430, as shown in FIGS. 6 and 7 respectively. The mask 420 in FIG. 6 can be made with a non-carrier adhesive tape with top and bottom protective films and the mask 420 in FIG. 7 can be made with a double-sided adhesive tape with top and bottom protective films. The protective films are made of plastics such as PET, APET, PVC etc. which are hydrophobic or have low surface energy, which make a good mask surface. These protective films can undergo surface treatments to enhance their hydrophobicity. The non-hydrophobic layers in FIGS. 6 and 7 can be made of a layer of acrylic adhesive.

### Example 4

### Manufacturing of Dissolvable Microneedle Patches

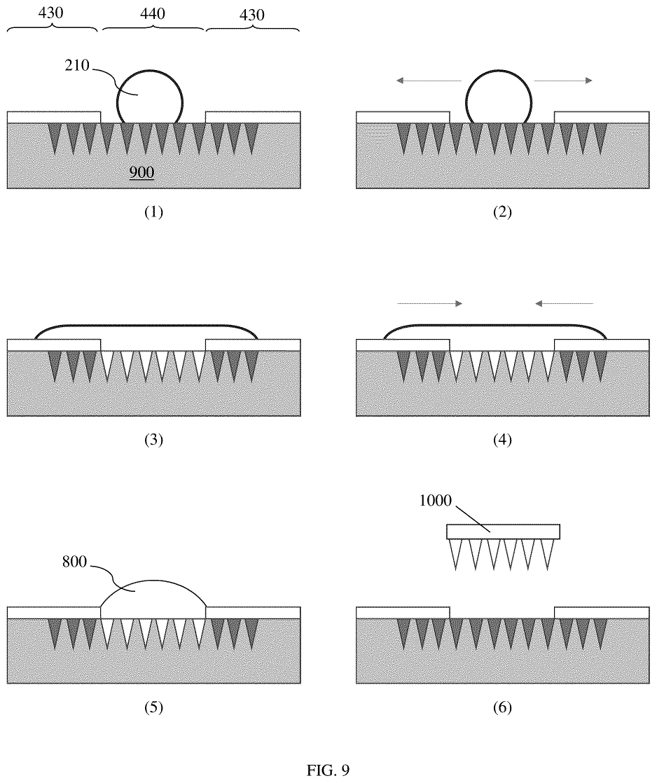

In this example, a mould system from the second preferred embodiment will be used to manufacture dissolvable microneedles. Other preferred embodiments are used in the same way. The manufacturing steps are in the following paragraphs and also shown in FIG. 9.

Firstly, a mould system 900 of the second preferred embodiment is provided. The mould system 900 is put in a vacuum chamber and subjected to vacuum pressure for a length of time, for example but not limited to −0.7 bar to −1 bar and 1 to 60 minutes respectively. After the vacuum treatment (this step is not shown in the figure), the mould system 900 is removed from the vacuum chamber and placed under atmospheric pressure (Step 1).

Secondly, a material solution 210, which is separately prepared, is dispensed on the mould under atmospheric pressure as soon as the mould system 900 is taken out of the vacuum chamber. The material solution 210 is dispensed within the through hole 440 (Step 2), which is also on the microneedle cavities 110 of the flat silicone rubber mould 100.

Thirdly, the material solution 210 is spread out to cover the entire through hole 440 by pushing the liquid beyond the boundary of the through hole 440, as shown in FIG. 9 (Step 3). As explained earlier, due to the hydrophobicity of the mask surface 430, the material solution 210 has a prevailing surface tension force which tends to minimize its surface area by pulling back the liquid (Step 4) (instead of spreading out) and forming a meniscus 800 around the boundary of the through hole 440 (Step 5).

Fourthly, the meniscus 800 is dried in room temperature which forms a dissolvable microneedle patch 1000 without flashing or burr (Step 6).

What is claimed is:

1. A mould system for manufacturing dissolvable microneedle patches, comprising:
(a) a flat silicone rubber mould, comprising a plurality of microneedle cavities on one of its surfaces; and
(b) a mask, comprising a mask surface and at least one through hole having the shape of the dissolvable microneedle patch;
wherein the mask is attached to the flat silicone rubber mould via the surface with microneedle cavities.

2. A mould system of claim 1, wherein the mask is thin and having thickness from 0.01 mm to 0.3 mm.

3. A mould system of claim 1, wherein the mask has the same thickness as the dissolvable microneedle patch.

4. A mould system of claim 1, wherein the mask surface is hydrophobic.

5. A mould system of claim 1, wherein the mask surface is coated with hydrophobic material.

6. A mould system of claims 1-3, wherein the mask is multi-layered and is constituted by:
(a) a top hydrophobic layer; and
(b) a bottom non-hydrophobic layer
wherein the bottom non-hydrophobic layer is attached to the flat silicone rubber mould via one of its surfaces comprising a plurality of microneedle cavities.

7. A mould system of claims 1-3, wherein the mask is multi-layered and is constituted by:
(c) a central carrier layer;
(d) a pair of top and bottom non-hydrophobic layers sandwiching the central carrier layer; and
(e) a top hydrophobic layer disposed on top of the top hydrophobic layer,
wherein the bottom non hydrophobic layer is attached to the flat silicone rubber mould via one of its surfaces comprising a plurality of microneedle cavities.

8. A mould system of claims 6 and 7, wherein the non-hydrophobic layer is made of acrylic adhesive.

9. A mould system of claims 6 and 7, wherein the thickness of the mask is between 0.01-0.30 mm.

10. A method of manufacturing dissolving microneedle patches, comprising:
(a) providing a mould system of claims 1, 6 or 7 and subjected the mould system to a vacuum pressure for a length of time;
(b) preparing a material solution;
(c) dispensing the material solution in the through holes of the mask of the mould system at atmospheric pressure;
(d) spreading the material solution to form a meniscus at the boundary of the through holes; and
(e) drying the material solution to form dissolvable microneedle patches.
